(19) United States
(12) Patent Application Publication
(10) Pub. No.: US 2026/0158220 A1
(43) Pub. Date: Jun. 11, 2026

(54) Medicament delivery device with click generator

(71) Applicant: SHL Medical AG, Zug (CH)

(72) Inventors: Daniel Carlsson, Nacka Strand (SE); Daniel Säll, Nacka Strand (SE)

(21) Appl. No.: 18/708,025

(22) PCT Filed: Oct. 26, 2022
(86) PCT No.: PCT/EP2022/079906

§ 371 Date: May 7, 2024

(30) Foreign Application Priority Data
Nov. 9, 2021 (EP) 21207277.1

(51) Int. Cl. A61M 5/20

(52) U.S. Cl. CPC A61M 5/2033; A61M 2205/581

(57) ABSTRACT

The present disclosure provides a power pack for a medicament delivery device, including a tubular body, a plunger rod, a power spring and a click generator. The click generator includes a flexible arm. The flexible arm at least partially extends radially inwards such that when the power spring expands during activation of the power pack, some coil sections of the power spring and the flexible arm repeatedly collide when the at least some coils move in a proximal direction, causing a repeated bending and release of the flexible arm. A material of the flexible arm has a stiffness high enough such that the repeated colliding of the at least some coil sections and the flexible arm, and/or the repeated bending and release of the flexible arm, generates a plurality of clicking sounds.

### CROSS-REFERENCE TO RELATED APPLICATIONS

The present application is a U.S. National Phase Application pursuant to 35 U.S.C. § 371 of International Application No. PCT/EP2022/079906 filed Oct. 26, 2022, which claims priority to European Patent Application No. 21207277.1 filed Nov. 9, 2021. The entire disclosure contents of these applications are herewith incorporated by reference into the present application.

### TECHNICAL FIELD

The present disclosure relates to the field of medicament delivery devices. In particular, the present disclosure relates to a power pack for such a device having a click generator.

### BACKGROUND

Autoinjectors and other types of medicament delivery devices are designed to allow for a user (e.g. a patient) to perform self-administration of a dose of a particular medicament in a controlled way. After positioning the device at the site of the body where the injection is desired, the user activates the device by pushing a release button. Pushing the release button releases a pre-compressed power spring which, when released, in turn drives a plunger rod towards the proximal end of the autoinjector. The plunger rod comes into contact with a syringe in which the medicament to be delivered is stored, and the movement of the plunger rod forces the medicament out of the syringe and into the injection site. In some autoinjectors, the movement of the plunger rod is also responsible for first inserting the needle into the bodily tissue at the injection site.

To help the user of the medicament delivery device, it can be desirable if the medicament delivery device provides some kind of feedback (such as visual feedback, tactical feedback and/or audible feedback) to the user during the injection process. For example, it may be desirable to indicate to the user whether the plunger rod is moving, and whether the injection is thus still ongoing.

To allow for a larger injection force, the power spring can be arranged on the outside of the plunger rod. This can allow to use a power spring with a larger diameter, without making the diameter of the combined plunger rod and power spring too large to fit within the syringe. However, with the power spring arranged on the outside of the plunger rod, access to the plunger rod is limited and including a feedback mechanism coupled to the plunger rod can thus be difficult.

### SUMMARY

To at least partially solve the above identified problem with providing feedback to the user during the injection process, the present disclosure provides an improved power pack, a click generator for such a power pack, and an improved medicament delivery device, as defined in the accompanying independent claims. Various alternative embodiments are defined in the dependent claims.

According to a first aspect of the present disclosure, a power pack for a medicament delivery device is provided. The power pack includes a tubular body extending along a longitudinal axis from a proximal end to a distal end. The tubular body includes an inner tubular section which is arranged fixed relative to the tubular body and which also extends along the longitudinal axis. The power pack further includes a plunger rod at least partially arranged within the inner tubular section and movable relative to the inner tubular section along the longitudinal axis. The power pack further includes a power spring. The power spring includes a plurality of coil sections. The power spring is at least partially arranged within the inner tubular section and expandable along the longitudinal axis. In a compressed state, the power spring (and the plurality of coil sections) at least partially surrounds the plunger rod (e.g., the power spring and plunger rod may be coaxially arranged such that the coil sections of the power spring encircle the plunger rod) such that the power spring, when released and expanding along the longitudinal axis during activation of the power pack, drives the plunger rod along the longitudinal axis towards the proximal end. The power pack further includes a click generator mounted to the tubular body. The click generator includes a flexible arm. The flexible arm at least partially extends radially inwards long enough towards the longitudinal axis such that when the power spring expands (during its release), at least some coil sections of the plurality of coil sections of the power spring and the flexible arm repeatedly collide when the at least some coils move towards the proximal end, thereby causing a repeated bending and release of the flexible arm. A material of the flexible arm has a stiffness high enough such that the repeated colliding of the at least some coils and the flexible arm, and/or the repeated bending and release of the flexible arm, generates a plurality of clicking sounds. In some embodiments, it may for example be only a) a moving coil section hitting the flexible arm in its relaxed (that is, unbent) position that generates a clicking sound. In some embodiments, it may for example be only b) the flexible arm hitting a coil section during release/spring-back of the flexible arm that generates the clicking sound. In some embodiments, it may be only c) the bending and release of the flexible arm itself that generates the clicking sound. In some embodiments, it may be any combination of these three ways of sound generation, such as only a) and b), only a) and c), only b) and c), or all of a), b) and c), which is responsible for generating the plurality of clicking sounds.

The present disclosure of the power pack according to the first aspect improves upon existing technology in that the envisaged click generator provides a convenient way of creating (audible) feedback to the user during the injection process, even with the power spring arranged on the outside of the plunger rod. This can help to reduce uncertainty about whether the injection has started, whether the injection is ongoing, or whether the injection has finished. Overall, the present disclosure provides an improved way of providing the user with information to assess whether the medicament delivery device is working properly. For example, by waiting until the clicking sounds have stopped, the user can be made confident that the injection is finished, and that the needle of the medicament delivery device can be safely withdrawn from the injection site. As will be described more in detail later herein, the click generator as envisaged herein can be added to existing medicament delivery devices and power packs, without requiring much or any modification of these already existing devices and components in order to achieve the improved functionality. Thus, the envisaged effect can be achieved without larger additional costs and/or larger changes to the manufacturing process.

In one or more embodiments of the power pack, the flexible arm may be made from sheet metal.

In one or more embodiments of the power pack, the material of the flexible arm may preferably be steel, such as e.g. spring steel or similar having the required stiffness. It is however envisaged to also use other materials, as long as the stiffness properties as described above are still present in such other materials.

In one or more embodiments of the power pack, a thickness of (e.g. an end of) the flexible arm may be smaller than a distance between neighboring coil sections in the compressed state of the power spring. This may cause each coil section moving past the flexible arm to generate a click, as (the end of) the flexible arm always fits between the coil sections, no matter how far the power spring has gotten in its expansion. This in turn may cause a continuous clicking during more or less the full injection process.

In one or more embodiments of the power pack, a thickness of (e.g. an end of) the flexible arm may be smaller than a distance between neighboring coil sections in the compressed state of the power spring. This may delay the generation of the clicking sounds until the power spring has expanded far enough such that the distance between the neighboring coil sections is larger than the thickness of the flexible arm. Such a delay may for example be useful in that the clicking sounds only occur towards the end of the injection process, thus alerting the user that it is soon possible to withdraw the injection needle from the body.

In one or more embodiments of the power pack, the click generator may be mounted to a proximal end of the inner tubular section.

In one or more embodiments of the power pack, a body of the click generator may be formed as a sleeve. The sleeve may be mounted to (e.g. around) the proximal end of the inner tubular section. The sleeve may for example be cylindrical, or have an oval, square or other shape which preferably matches the outer shape of the proximal end of the inner tubular section.

In one or more embodiments of the power pack, the sleeve of the body of the click generator may further include at least one flexible mounting tab bent radially inwards towards the longitudinal axis and pointing towards the proximal end of the tubular body. The proximal end of the inner tubular section may be made of (i.e. at least comprise) a plastic material. The sleeve may be mounted to the proximal end of the inner tubular section by the at least one mounting tab gripping into the plastic material (i.e. such that it prevents the sleeve from being pulled or pushed off the proximal end of the inner tubular section, towards the proximal end of the tubular body).

In one or more embodiments of the power pack, the power pack may further include a release button arranged within the tubular body and movable relative to the tubular body along the longitudinal axis (e.g. movable at least towards the proximal end of the tubular body). The release button may be configured to, if pushed along the longitudinal axis towards the proximal end, cause the activation of the power pack by releasing the power spring.

In one or more embodiments of the power pack, the inner tubular section may further include a distal flexible arm. The distal flexible arm of the inner tubular section may be configured to abut a distal rim of the plunger rod to block the power spring from being released and in particular the plunger rod from moving along the longitudinal axis towards the proximal end. The release button may further include a protrusion extending from a distal end of the release button towards the proximal end of the tubular body. This protrusion may be configured to, as a result of pushing the release button along the longitudinal axis towards the proximal end (of the tubular body), abut a distal surface of the flexible arm of the inner tubular section and cause a bending of the flexible arm of the inner tubular section radially outwards and away from the longitudinal axis. This bending may cause the activation of the power pack by releasing the power spring (and activating the power pack and the medicament delivery device) by preventing the blocking of the power spring from being released and in particular the plunger rod from moving along the longitudinal axis towards the proximal end.

In one or more embodiments of the power pack, during expansion of the power spring, a proximal end of the power spring may (be configured to) abut a proximal rim of the plunger rod, thus driving the plunger rod along the longitudinal axis towards the proximal end of the power pack.

According to a second aspect of the present disclosure, a click generator for a power pack for a medicament delivery device is provided. The click generator extends along a longitudinal axis from a proximal end to a distal end. The click generator includes a body extending along the longitudinal axis. The body is formed as a sleeve. The body may for example be configured such that it can be mounted to an end of an inner tubular section of a body of the power pack, as described earlier herein. The click generator further includes a flexible arm arranged at the proximal end and at least partially extending radially inwards towards the longitudinal axis. The flexible arm may for example form part of an outer shell of the body. A material of the flexible arm has a stiffness high enough such that a repeated colliding of the flexible arm and coil sections of a power spring of the power pack, and/or a repeated bending and release of the flexible arm, causes generation of a plurality of clicking sounds. This envisaged click generator has, for example, the benefit of being retrofittable to already existing power packs, such that the envisaged clicking sounds can be generated without larger or any modifications to the other parts and components of the already existing power packs.

In one or more embodiments of the click generator, the flexible arm is made from sheet metal.

In one or more embodiments of the click generator, the material of the flexible arm is preferably steel.

In one or more embodiments of the click generator, for mounting of the sleeve to the end of the inner tubular section of the body of the power pack, the cylindrical sleeve may further include at least one flexible mounting tab. The at least one flexible mounting tab may be bent radially inwards towards the longitudinal axis and pointing towards the proximal end. Such a construction may allow the sleeve of the click generator to be pushed onto the end of the inner tubular section (as the mounting tab is flexible), while the mounting tab may grip into the end of the inner tubular section if an attempt is made to pull or push the sleeve and the click generator off the end of the inner tubular section, thereby providing a securing/fixing of the click generator to the inner tubular section. In particular, such a construction is especially suitable if the click generator is provided for retrofitting to already existing power packs. Even if not retrofitted, but instead provided during manufacturing of the power pack, the envisaged mounting solution of the click generator provides an easy way of attaching the click generator to the end of the inner tubular section, requiring no additional elements such as adhesives or similar.

According to a fourth aspect of the present disclosure, a medicament delivery device is also provided. The medicament delivery device includes a power pack according to the first aspect.

Other objects and advantages of the present disclosure will be apparent from the following detailed description, the drawings and the claims. Within the scope of the present disclosure, it is envisaged that all features and advantages described with reference to e.g. power pack of the first aspect are relevant for, apply to, and may be used in combination with also any feature and advantage described with reference to the tubular body of the second aspect, the release button of the third aspect, and the medicament delivery device of the fourth aspect, and vice versa.

### BRIEF DESCRIPTION OF THE DRAWINGS

Exemplifying embodiments will now be described below with reference to the accompanying drawings, in which:

FIG. 1A schematically illustrates a perspective view of an embodiment of a medicament delivery device according to the present disclosure;

FIGS. 1B-1E schematically illustrate cross-sections of various embodiments of a power pack and click generator according to the present disclosure, and

FIG. 1F schematically illustrates a perspective view of an embodiment of a click generator according to the present disclosure.

In the drawings, like reference numerals will be used for like elements unless stated otherwise. Unless explicitly stated to the contrary, the drawings show only such elements that are necessary to illustrate the example embodiments, while other elements, in the interest of clarity, may be omitted or merely suggested. As illustrated in the Figures, the (absolute or relative) sizes of elements and regions may be exaggerated or understated vis-à-vis their true values for illustrative purposes and, thus, are provided to illustrate the general structures of the embodiments.

### DETAILED DESCRIPTION

Exemplifying embodiments of a medicament delivery device, a power pack, a tubular body, and a release button according to the present disclosure will now be described more fully hereinafter with reference to the accompanying drawings. The drawings show currently preferred embodiments, but the invention may, however, be embodied in many different forms and should not be construed as limited to the embodiments set forth herein; rather, these embodiments are provided for thoroughness and completeness, and fully convey the scope of the present disclosure to the skilled person.

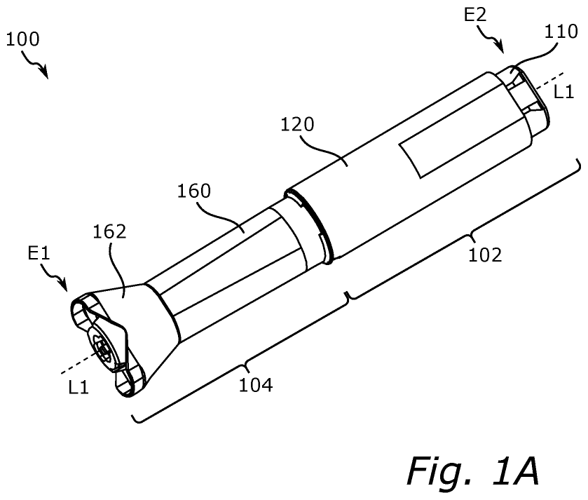

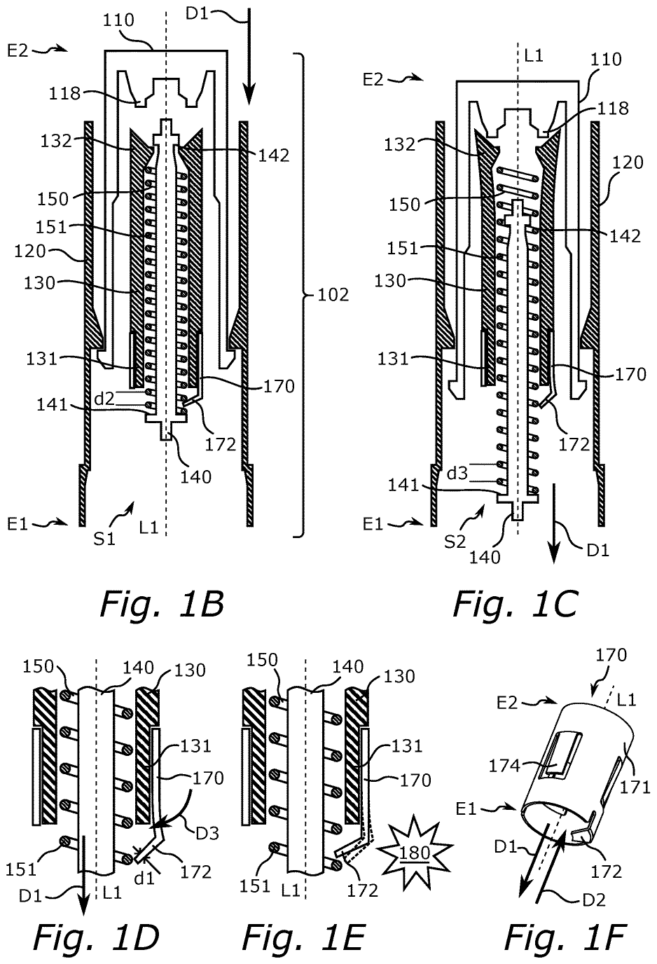

FIG. 1A schematically illustrates a medicament delivery device 100 according to one embodiment of the present disclosure. Hereinafter, the terms “medicament delivery device” and “device” will be used interchangeably.

The device 100 can for example be manufactured as two sub-assemblies, namely a so-called power pack 102 and a so-called front assembly 104, which are joined together during final assembly of the device 100. In other embodiments, it is envisaged that the device 100 may arrive fully assembled from the factory, with the power pack 102 and the front assembly 104 already joined together as illustrated in FIG. 1A. The power pack 102 and the front assembly 104 are arranged such that they both extend along a longitudinal axis L1, from a proximal end E1 to a distal end E2. The power pack 102 includes a tubular body 120 and a release button 110, both extending along the longitudinal axis L1. The release button 110 is at least partially arranged within the tubular body 120 and is movable along the longitudinal axis L1. Here, “movable” implies that the release button can, if not blocked by any other component of the power pack, be pushed at least in a proximal direction D1 relative to the tubular body 120. Such pushing of the release button 110 can be achieved e.g. by a user using a finger, such as a thumb, to apply a force on the release button in the proximal direction.

Although not explicitly indicated in FIG. 1A, it is to be understood that each one of the power pack 102 and the front assembly 104 can also be considered as having its own proximal and distal ends, and that each one of the power pack 102 and the front assembly 104 can also be considered as extending along its own longitudinal axis. When arranged as in FIG. 1A, the longitudinal axis of each sub-assembly aligns with the longitudinal axis L1 of the device as a whole. Likewise, the distal end of the power pack is also the distal end E2 of the device 100, while the proximal end of the front assembly 104 is the proximal end E1 of the device 100. The proximal end of the power pack 102 can be understood as the end of the power pack 102 being closest to the front assembly 104 and, similarly, the distal end of the front assembly 104 can be understood as the end of the front assembly 104 being closest to the release button 102.

Similarly, each particular component of the power pack 102 and the front assembly 104 can be defined as having its own proximal and distal ends, and also its own longitudinal axis. When so doing, it can be assumed that the components are all arranged as indicated in FIG. 1A. The references L1, E1 and E2 will be used throughout the present disclosure to indicate such longitudinal axis, proximal end distal ends of each component, as needed. Even if the device 100 is fully assembled, it is envisaged that the various proximal and distal ends of the various components are still as they were before the device 100 was fully assembled, as illustrated in FIG. 1A. The device 100 can be fully assembled by e.g. pushing the distal end of the front assembly 104 into the proximal end of the power pack 102.

The front assembly 104 can optionally include a protective cap 162, which functions e.g. to protect a needle (not shown) of the front assembly 104 from being accessible by a user of the device 100. When the device 100 is to be used, the protective cap 164 can first be removed by the user. In some embodiments, the device 100 can be configured (using e.g. a rotator, also not shown) such that even with the protective cap 162 removed, the device 100 must first be pressed against the injection site before the release button 110 can be used to activate the power pack and the device, and cause the injection of the medicament into the body of the user.

The various components of the device 100 related to the power pack 104, as envisaged by the present disclosure, will now be described in more detail with reference to FIGS. 1B-1F.

FIG. 1B schematically illustrates a cross-section of an embodiment of the power pack 102, where the power pack 102 is not yet assembled with the front assembly. The power pack includes the tubular body 120. The tubular body 120 extends along the longitudinal axis L1 from a proximal end E1 to a distal end E2. The tubular body 120 further includes an inner tubular section 130 which is arranged fixed relative to the tubular body 120 and which also extends along the longitudinal axis L1. It is envisaged that, to fix the inner tubular section 130 with respect to the tubular body 120, one or more support structures (not shown) may for example extend from an inside wall of the tubular body 120 to the inner tubular section 130.

The power pack 120 further includes a plunger rod 140 and a power spring 150, which are both at least partially arranged within the inner tubular section 130. Here, “at least partially” is to be understood that there may be some parts of the power spring 150 and the plunger rod 140 that extends out of the inner tubular section 130, e.g. either at one or both of a proximal end and a distal end of the inner tubular section 130. The power spring 150 includes a plurality of coil sections 151. Each coil section 151 may for example be considered as one complete turn of wire coiled to create the power spring 150. The power spring 150 is arranged such that it can, if not intentionally blocked by any other components of the power spring, expand along the longitudinal axis L1. In the particular embodiment illustrated in FIG. 1B, the power spring 150 is in particular arranged to expand towards the proximal end E1 while it can have no or little expansion in the opposite direction, towards the distal end E2.

In FIG. 1B, the power spring 150 is in a compressed state S1. Here, “compressed” does not necessarily mean fully compressed, but at least compressed enough such that the power spring 150 will expand as described above when the compression is released. In the compressed state S1, the power spring 150 at least partially surrounds the plunger rod 140 such that the power spring 150, when released and expanding (as will be described later herein with reference to FIG. 1C) along the longitudinal axis L1, can drive the plunger rod 140 along the longitudinal axis L1 towards the proximal end E1. In the particular embodiment illustrated in FIG. 1B, this is achieved by a proximal end of the power spring 150 abutting a proximal rim 141 of the plunger rod, while a distal end of the power spring 150 abuts e.g. a distal section of the inner tubular section 130.

In particular, the power pack 102 as envisaged herein also includes a click generator 170. The click generator 170 is mounted to the tubular body 120. Here, this is achieved by mounting the click generator 170 to a proximal end 131 of the inner tubular structure 130, but it is envisaged that other ways of mounting the click generator 170 to the tubular body 120 are also possible. For example, in some embodiments, the click generator 170 may be mounted e.g. to an inside wall of the tubular body 120. It is envisaged that any mounting of the click generator 170 such that it does not move with the power spring 150 (during expansion), and such that it comes into contact with the power spring 150, is suitable to achieve the envisaged effect.

The click generator 170 includes a flexible arm 172 which at least partially extends radially inwards, towards the longitudinal axis L1. This extension of the flexible arm 172 is in particular long enough such that at least an end of the flexible arm 172 reaches and preferably passes (in a radially inwards direction) the radial position of the plurality of coil sections 151 of the power spring 150. Phrased differently, a distance from the longitudinal axis L1 to the end of the flexible arm 172 is preferably equal or smaller than radius of the power spring 150. As a result of such an arrangement, when the power spring expands, at least some coil sections of the plurality of coil sections 151 and the flexible arm 172 will repeatedly collide when the at least some coil sections move towards the proximal end E1. This will cause a repeated bending and releasing of the flexible arm 172. By making the flexible arm 172 of (or at least comprising) a material which has a high enough stiffness, the repeated colliding of the at least some coil sections and the flexible arm 172, and/or the repeated bending and releasing of the flexible arm 172, will generate a plurality of clicking sounds, and thus help to provide the desired (audible) feedback to the user about the current state of the injection process. The flexible arm can for example be made from sheet metal, such as for example steel or spring steel, as long as the stiffness is sufficient to generate the clicking sounds when the arm is for example repeatedly hit/impacted by moving coil sections 151, when a coil section is repeatedly hit/impacted by a coil section springing back after being bent, and/or due to the repeated bending and release of the flexible arm 172 itself. Other materials, such as e.g. various plastics and similar, may also be suitable as long as the stiffness requirement is fulfilled.

In the particular embodiment illustrated in FIG. 1B, a thickness of the flexible arm is such that it is smaller than a spacing d2 between nearby coil sections, even when the power spring 150 is in the compressed state S2. As described earlier herein, this will lead to an immediate generation of clicking sounds once the power spring 150 is released, and the clicking sounds will continue until the injection process has been completed. The exact sequence of clicking sounds generated, e.g. a time distance between successive clicks, can depend on e.g. the spring constant of the power spring, the distance d2, and other such parameters. In other embodiments, it may be envisaged that when the power spring 150 is compressed, the separation d2 in distance between nearby coil sections 151 is smaller than the thickness of (at least the end of) the flexible arm 172. If this is the case, clicking sounds will be generated only after the power spring 150 has expanded sufficiently for the distance between the coils to become larger than the thickness of the flexible arm 172. As described earlier herein, this may cause a clicking sound generation only towards e.g. an end of the injection process.

The click generator 170 may for example be fastened to the end of the inner tubular section 130 by using an adhesive, tape, or other means of fastening. As will be described later herein with reference to FIG. 1F, other means of fastening/fixing the click generator 170 requiring no such adhesives are also envisaged. In particular, it is envisaged that the click generator 170 is preferably manufactured as a separate component that may be mounted to e.g. the proximal end 131 of the inner tubular section 130 after e.g. the power spring 150 and plunger rod 140 are inserted into the inner tubular section 130. The shape of the flexible arm 172 is thus preferably such that it allows the click generator 170 to be pushed over the power spring 150 and plunger rod 140 in a direction towards the distal end E2, due to the flexing of the flexible arm 172.

The power pack 102 can also include a release button 110. The release button 110 is at least partially arranged within the tubular body 120 and is movable (with respect to the tubular body 120) along the longitudinal direction L1 of the tubular body 120. In the position of the various components shown in FIG. 1B, the release button 110 does not yet interact with any other components of a release mechanism for the power spring 150 and plunger rod 140.

Such a release mechanism preferably provides two things. First, it should be able to hold/lock the power spring 150 compressed and the plunger rod 140 still before the release button 110 is pushed. Second, it should be able to release such a hold of the power spring 150 and plunger rod 140 once the release button 110 is pushed, such that the power pack 102 is then activated. One envisaged way of holding/locking the plunger rod 140 and the power spring 150 before the activation of the power pack 102 is illustrated in FIG. 1B and described as follows. A distal end of the power spring 150 abuts e.g. part of the inner tubular section 130, while a proximal end of the power spring 150 abuts a proximal rim 141 of the plunger rod 140. With the release button 110 in the position as shown in FIG. 1B, the power spring 150 is at least partially compressed and exerts a force on the plunger rod 140 in the proximal direction D1 by the proximal end of the power spring 150 pressing against the proximal rim 141 of the plunger rod 140. To keep the plunger rod 140 from being propelled/driven by the power spring 150 in the proximal direction D1, a distal end of the inner tubular section 130 further includes a (distal) flexible arm 132. As shown in FIG. 1B, part of the flexible arm 132 abuts a corresponding distal rim 142 of the plunger rod 140, which blocks the plunger rod 140 from moving in the distal direction D2. As will be described in more detail later with reference to FIG. 1C, the holding/blocking of the plunger rod 140 caused by the flexible arm 132 and the distal rim 142 can be released by pressing of the release button 110 in the proximal direction D1.

In all the figures herein illustrating cross-sections, it should be noted that although some features are shown in a same cross-sectional plane, that must not necessarily be the case. For example, in the power pack 102 illustrated in FIG. 1B, it may for example be envisaged that the features related to the activation/locking of the plunger rod 140 and power spring 150, including e.g. the flexible arm 132 of the inner tubular section 130, are instead arranged such that they would only show up in another cross-sectional plane, e.g. a cross-sectional plane perpendicular or otherwise angled with respect to the cross-sectional plane shown in FIG. 1B. However, for illustrative purposes only, the components described are assumed to be arranged such that a single cross-section can illustrate all described features.

FIG. 1C schematically illustrates a cross-section of the same power pack 102 as in FIG. 1B, but in another state S2 wherein the release and expansion of the power spring 150 towards the proximal end E1 (i.e. in the proximal direction D1) has started.

With the release button 110 pushed along the longitudinal axis L1 towards the proximal end E1, as shown in FIG. 1C, the release button 110 causes an activation of the power pack 102 as follows. At a distal end of an inside of the release button 110, there is provided a protrusion 118 which extends from the distal end of the release button 110 towards the proximal end El of the tubular body 120. When the release button 110 is pushed such as shown in FIG. 1C, the protrusion 118 abuts a distal surface of the flexible arm 132 of the inner tubular section 130, and causes a bending of the flexible arm 132 radially outwards. When the flexible arm 132 is bent radially outwards, it is separated from the distal rim 142 of the plunger rod 140 and can no longer prevent/block the plunger rod 140 from being propelled/driven in the proximal direction D1 by the proximal force applied on the proximal rim 141 of the plunger rod 140 by the at least partially compressed power spring 150. As the power spring 150 expands, the plunger rod 140 is accelerated and moved along the longitudinal axis L1 in the proximal direction D1. This completes the activation of the power pack. The plunger rod 140 may then, in turn, reach and interact with e.g. a syringe/ampulla located in the front assembly, and cause an expulsion of a medicament from the syringe/ampulla. The movement of the plunger rod 140 may also, in some embodiments, be responsible for first causing a movement of a needle of the front assembly in the proximal direction, such that the needle is automatically inserted into the delivery site of the user before the medicament is expelled from the syringe/ampulla.

It should be noted that the features related to the activation of the power pack 102, i.e. the holding and release of the power spring 150 and plunger rod 140 (e.g. the release button 110, the protrusion 118, the flexible arm 132 of inner tubular section 130, the plunger rod 140 and its various rims 141 and 142, etc.) and their interactions are here discussed only for reasons of completeness, even though their exact function, arrangement, configuration and similar is not directly relevant to the core of the present disclosure, which is the provisioning of audible feedback to the user during the injection process of the device.

In particular, and as already touched upon earlier herein, simultaneously with activating the power pack 102 by pressing on the release button 110, the state S2 including movement and expansion of the power spring 150 will cause at least some of the plurality of coil sections 151 and the flexible arm 172 to repeatedly collide. For each such coil section coming into contact with, and finally moving past, the (end of the) flexible arm 172, the flexible arm 172 is bent radially outwards (i.e. away from the longitudinal axis), before it (the flexible arm 172) then springs back to its original position. This can generate a repeated generation of clicking sounds. More generally, it is envisaged herein that a clicking sound may be generated when the flexible arm 172 is still and is hit/impacted by a moving coil section, when the flexible arm 172 is moving when returning back to its original position and hits the next incoming coil section, due to the bending and release of the flexible arm 172 itself, and/or by a combination of these various situations. As indicated in FIG. 1C, a distance d3 between neighboring coil sections during the state S2 is larger than the corresponding distance d2 during the state S1 (where the power spring 150 is compressed), which can be used to tailor the sequence of clicking sounds as described earlier. Either the clicking sounds are generated during the whole expansion of the power spring (such as when d1<d2), or the clicking sounds are generated only during part of the expansion of the power spring, in particular a later part of the expansion (such as when d2<d1<d3).

FIGS. 1D and 1E show in more detail the bending and releasing of the flexible arm 172 as the flexible arm 172 and the coil sections 151 of the power spring 150 repeatedly collide, as the coil sections 151 move in the proximal direction D1. FIGS. 1D and 1E only show the parts of the power pack 102 which are directly responsible for forming the desired audible feedback to the user, and which thus forms the core of the envisaged solution.

In FIG. 1D, the click generator 170 is mounted to a proximal end 131 of the inner tubular section 130. A particular coil section 151 of the power spring has recently hit/impacted the end of the flexible arm 172 of the click generator (which flexible arm has a thickness d1 smaller than a current distance between the coil section 151 and the one closest in a distal direction). As the coil section 151 has continued to move in the proximal direction D1, the surface of the coil section 151 recently hitting/impacting the flexible arm 172 has forced the flexible arm 172 to bend radially outwards away from the longitudinal axis L1. The position of the coil section 151 is such that if it moves just a bit more in the distal direction D1, it will lose contact with the flexible arm 172 and the flexible arm 172 will respond by springing back towards its initial position, i.e. moving in the “spring-back direction” indicated by the arrow D3. In some embodiments, the coil section 151 hitting the flexible arm 172 is sufficient to cause the generation of a clicking sound. In other embodiments, in addition or alternatively, it can be the coil section 151 springing-back and thereby hitting the next incoming coil section which generates the clicking sound, and/or it can be e.g. the bending and subsequent spring-back (release) of the flexible arm 172 itself which causes such a clicking sound (as illustrated in FIG. 1E).

In FIG. 1E, the coil section 151 has now moved sufficiently far in the proximal direction D1 to lose contact with the flexible arm 172, and the flexible arm 172 has therefore sprung back into its initial position. As the stiffness of the flexible arm 172 (that is, a material forming the whole of, or at least part of the flexible arm) is sufficiently high, the bending and subsequent release/spring-back of the flexible arm 172 causes a clicking sound 180 which is audible to the user (assuming of course that the hearing capability of the user is sufficiently functional, i.e. “normal”). Once the next coil section of the power spring 150 passes the flexible arm 172 in a same way, a new clicking sound 180 will be generated. As described above, and although not illustrated explicitly in FIG. 1E, it may also be such that if the power spring 150 is expanding fast enough in the proximal direction, the flexible arm 172 may not be able to return back to its original position before it collide with the next coil section of the power spring 150. This situation (where the flexible arm 172 is not just passive but actively hits a coil during spring-back/release) may also cause the clicking sound 180 to be generated.

A particular embodiment of a click generator according to the present disclosure will now be described in more detail with reference to FIG. 1F.

FIG. 1F schematically illustrates a perspective view of a click generator 170. The click generator 170 may for example be similar or equal to the click generator 170 described with reference to FIGS. 1B-1E. The click generator 170 extends along a longitudinal direction L1 from a proximal end El to a distal end D2. The click generator 170 has a body 171 which is shaped like a sleeve. In this particular embodiment, the sleeve of the body 171 is cylindrical, but it is envisaged that also other shapes may be used. In particular, the shape of the sleeve may be such that it matches an outer shape of e.g. the end 131 of the inner tubular section 130 at which the click generator 170 is to be mounted.

The click generator 170 includes a flexible arm 172 which is arranged at the proximal end E1. The flexible arm 172 at least partially extends radially inwards towards the longitudinal axis L1 (if the geometry of the click generator 170 is not circular, the term “radially” may e.g. be replaced with “laterally”, e.g. meaning that something points either outwards and away from the longitudinal axis L1, or inwards and towards the longitudinal axis L1). The exact length of the radially inwards extending part of the flexible arm 172 can be adapted to the size/diameter of the power spring with which it is to interact, although it is important that it extends long enough to at least come into contact with the coil sections of the power spring as the power spring expands). As before, a material of the flexible arm has a stiffness high enough such that a repeated colliding of the flexible arm 172 and some coil sections of the power spring, and/or a repeated bending and releasing of the flexible arm 172 (as caused by interaction with the coil sections of the power spring) causes generation of a plurality of clicking sounds. The flexible arm 172 may for example, as shown in FIG. 1F, be formed as part of an outer shell (that is, as part of the sleeve) of the body 171 of the click generator 170. In other embodiments, it is envisaged that the flexible arm 172 may for example be a separate component which is mounted to the body 171 of the click generator 170, such that the body 171 is not necessarily also formed by a material having the stiffness properties required for the flexible arm 172.

In the particular embodiment of the click generator 170 illustrated in FIG. 1F, the sleeve of the body 171 of the click generator 170 further includes at least one flexible mounting tab 174. The flexible mounting tab 174 is formed by cutting out parts of the shell/sleeve of the body 171, and is bent radially inwards towards the longitudinal axis L1, and such that it points towards the proximal end E1. As hinted at in FIG. 1F, there may be more than one mounting tab 174 provided, such as e.g. two, three, four or more.

If the flexible mounting tab 174 is e.g. made from a material harder than the material of the end of the inner tubular section at which the click generator 170 is to be mounted, the flexible mounting tab 174 (due to its flexibility and orientation) can allow the sleeve to be pushed onto the inner tubular section in the distal direction D2. However, a subsequent attempt to remove the sleeve in the proximal direction D1 can be blocked, as the flexible mounting tab 174 (due to it extending radially inwards) can dig into the material of the inner tubular section, thereby fixing the sleeve and the click generator 170 to the inner tubular section.

In summary of FIGS. 1A-1F, it is concluded that the various components of the device 100, and in particular of the power pack 102 (such as the click generator 170) as envisaged herein are configured to interact such that: i) the flexible arm 172 of the click generator 170 is arranged such that it comes into contact with the coil sections 151 of the power spring 150; ii) as the power spring 150 expands, the mechanical interaction of the coil sections 151 with the flexible arm 172 causes the flexible arm 172 to repeatedly bend and release, and thus generates a plurality of clicking sounds (due to the stiffness of the flexible arm 172), and iii) this provides a convenient way of generating audible feedback to the user about the state of the injection process, even though the plunger rod 140 is within the power spring 150 and not directly accessible. These features together provide an improved device and power pack, wherein the uncertainty for the user about the current state of the injection process is reduced or even removed.

Although not explicitly disclosed in any drawings, or at least not explicitly further described in the description so far, it is herein envisaged that the medicament delivery device may also include additional components, including e.g. a delivery member, the protective cap for the delivery member, an ampulla for storing the dose of medicament, one or more guiding rods and or other structures for the arrangement of a delivery member cover, a rotator for blocking pushing of the release button before e.g. the delivery member cover is pressed against the injection site, etc. It is envisaged that any such components are not directly relevant for describing the core idea underlying the present disclosure (i.e. the generation of audible feedback), and any further illustration and/or discussion of such additional components has therefore, for reasons of clarity, been left out of the present disclosure.

Medicament delivery devices as envisaged herein may for example be autoinjectors containing insulin, allergy medicaments, epinephrine, migraine medicaments, atropine, or any other medicament/drug for which the use of a medicament delivery device as described herein is suitable. The device can e.g. be of a single-use type (i.e. disposable). Delivery members may include needles, parts of syringes, syrettes, or other suitable structures.

In the present disclosure, the term “longitudinal axis” of the device refers to an axis extending from a proximal end of the device to a distal end of the device, typically a central axis along the device in the direction of longest extension of the device. The term “distal end” refers to the part/end of the device, or the parts/ends of the members thereof, which under use of the device is/are located furthest away from the dose delivery/injection site. Correspondingly, the term “proximal end” refers to the part/end of the device, or the parts/ends of the members thereof, which under use of the device is/are located closest to the dose delivery/injection site. As described further above, this also apply to the various components of the device itself, such as the power pack, the front assembly, the release button, the tubular body of the power pack, the click generator, etc., which can all be considered as each having their own longitudinal axis (where applicable), and which, when assembled in the device, can all be considered as having also their own corresponding proximal and distal ends.

Although features and elements may be described above in particular combinations, each feature or element may be used alone without the other features and elements or in various combinations with or without other features and elements. Additionally, variations to the disclosed embodiments may be understood and effected by the skilled person in practicing the claimed invention, from a study of the drawings, the disclosure, and the appended claims.

In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

1-14. (canceled)

15. A power pack for a medicament delivery device, comprising:
a tubular body extending along a longitudinal axis from a proximal end to a distal end, the tubular body including an inner tubular section arranged fixed relative to the tubular body and also extending along the longitudinal axis;
a plunger rod at least partially arranged within the inner tubular section and movable relative to the inner tubular section along the longitudinal axis;
a power spring including a plurality of coil sections, the power spring being at least partially arranged within the inner tubular section and expandable along the longitudinal axis and, in a compressed state, at least partially surrounding the plunger rod such that the power spring, when released and expanding along the longitudinal axis during activation of the power pack, drives the plunger rod along the longitudinal axis towards the proximal end, and
a click generator mounted to the tubular body and including a flexible arm, the flexible arm at least partially extending radially inwards long enough towards the longitudinal axis such that when the power spring expands, at least some coil sections of the plurality of coil sections of the power spring and the flexible arm repeatedly collide when the at least some coil sections move towards the proximal end, thereby causing a repeated bending and release of the flexible arm,
wherein the flexible arm has a stiffness high enough such that the repeated colliding of the at least some coil sections and the flexible arm, and/or said repeated bending and release of the flexible arm, generates a plurality of clicking sounds.

16. The power pack according to claim 15, wherein the flexible arm is made from a sheet metal.

17. The power pack according to claim 15, wherein a thickness of the flexible arm is smaller than a distance between neighboring coil sections in the compressed state of the power spring.

18. The power pack according to claim 15, wherein a thickness of the flexible arm is greater than a distance between neighboring coil sections in the compressed state of the power spring, but smaller than a distance between neighboring coil sections during only part of the expansion of the power spring.

19. The power pack according to claim 15, wherein:
the click generator is mounted to a proximal end of the inner tubular section.

20. The power pack according to claim 19, wherein:
a body of the click generator is formed as a sleeve, and
the sleeve is mounted to the proximal end of the inner tubular section.

21. The power pack according to claim 20, wherein:
the sleeve of the body of the click generator further comprises at least one flexible mounting tab bent radially inwards towards the longitudinal axis and pointing towards the proximal end of the tubular body;
the proximal end of the inner tubular section is made of a plastic material, and
the sleeve is mounted to the proximal end of the inner tubular section by said at least one mounting tab gripping into said plastic material.

22. The power pack according to claim 15, further comprising:
a release button arranged within the tubular body and movable relative to the tubular body along the longitudinal axis, and configured to, if pushed along the longitudinal axis towards the proximal end, cause the activation of the power pack by releasing the power spring.

23. The power pack according to claim 22, wherein:
the inner tubular section includes a distal flexible arm configured to abut a distal rim of the plunger rod to block the power spring from being released and the plunger rod from moving along the longitudinal axis towards the proximal end, and
the release button includes a protrusion extending from a distal end of the release button towards the proximal end of the tubular body, and configured to, as a result of pushing the release button along the longitudinal axis towards the proximal end, abut a distal surface of the flexible arm of the inner tubular section and cause a bending of the flexible arm of the inner tubular section radially outwards and away from the longitudinal axis, thereby causing the activation of the power pack by preventing the blocking of the power spring from being released and the plunger rod from moving along the longitudinal axis towards the proximal end.

24. The power pack according to claim 15, wherein:
during the expansion of the power spring, a proximal end of the power spring abuts a proximal rim of the plunger rod, thus driving the plunger rod along the longitudinal axis towards the proximal end of the power pack.

25. A click generator for a power pack for a medicament delivery device, extending along a longitudinal axis from a proximal end to a distal end, and comprising:
a body extending along the longitudinal axis and formed as a sleeve, and
a flexible arm arranged at the proximal end and at least partially extending radially inwards towards the longitudinal axis,
wherein the flexible arm has a stiffness high enough such that a repeated colliding of the flexible arm and coil sections of a power spring of the power pack, and/or a repeated bending and release of the flexible arm, causes generation of a plurality of clicking sounds.

26. The click generator according to claim 25, wherein the flexible arm is made from a sheet metal.

27. The click generator according to claim 25, wherein:
the cylindrical sleeve further comprises at least one flexible mounting tab bent radially inwards towards the longitudinal axis and pointing towards the proximal end.

28. A medicament delivery device, comprising a power pack according to claim 15.
